(19) United States
(12) Patent Application Publication
(10) Pub. No.: US 2023/0063690 A1
(43) Pub. Date: Mar. 2, 2023

(54) COMPOSITIONS AND METHODS FOR REDUCING PAIN AND SYMPTOM(S) ASSOCIATED WITH SEIZURES, SLIGHT STROKES, AND RELATED ILLNESSES AND CONDITIONS

(71) Applicant: Rengen Atchana, East Orange, NJ (US)

(72) Inventor: Rengen Atchana, East Orange, NJ (US)

(21) Appl. No.: 17/411,057

(22) Filed: Aug. 25, 2021

(51) Int. Cl. A61K 36/185; A61P 25/04; A61K 47/44; A61P 25/08; A61P 9/10

(52) U.S. Cl. CPC A61K 36/185; A61P 25/04; A61K 47/44; A61P 25/08; A61P 9/10

(57) ABSTRACT

The present invention is directed to compositions for reducing pain, to compositions for reducing symptom(s) associated with seizures, slight strokes, and related illnesses and conditions, to methods for reducing pain, and to methods for reducing symptom(s) associated with seizures, slight strokes, and related illnesses and conditions. An embodiment of a composition mixture of ingredients for reducing pain and symptom(s) associated with seizures, slight strokes, and related illnesses and conditions includes marijuana, hand salve, healing ointment, water, and oil. An embodiment of a method for reducing pain and symptom(s) associated with seizures, slight strokes, and related illnesses and conditions includes the step of providing an effective amount of a composition for being applied externally onto affected body part(s) or area(s) of a subject or user.

### BACKGROUND OF THE INVENTION

### Field of the Invention

The present invention relates to compositions and methods for reducing pain (such as, but not limited to, body pain) and symptom(s) associated with seizures, slight strokes, and related illnesses and conditions.

Compositions and methods for reducing pain and symptom(s) associated with seizures, slight strokes, and related illnesses and conditions are known in the art.

The inventor of the present invention is unaware of inventions or patents, taken either singly or in combination, which are seen to describe the present invention as claimed.

### SUMMARY OF THE INVENTION

The present invention is directed to compositions for reducing pain (such as, but not limited to, body pain).

The present invention is directed to compositions for reducing symptom(s) associated with seizures, slight strokes, and related illnesses and conditions.

The present invention is directed to methods for reducing pain (such as, but not limited to, body pain).

The present invention is directed to methods for reducing symptom(s) associated with seizures, slight strokes, and related illnesses and conditions.

### DETAILED DESCRIPTION OF EMBODIMENTS

Hereinafter, exemplary embodiments of the present invention will be described in detail. However, the present invention is not limited to the embodiments disclosed below, but can be implemented in various forms. The following embodiments are described in order to enable those of ordinary skill in the art to embody and practice the present invention.

The inventor of the present invention focused on developing a composition(s) and a method(s) which could reduce pain (such as, but not limited to, body pain) and symptom(s) associated with seizures, slight strokes, and related illnesses and conditions, and confirmed that the composition(s) and method(s) of the present invention has an excellent effect on reducing pain (such as, but not limited to, body pain) and symptom(s) associated with seizures, slight strokes, and related illnesses and conditions, thereby completing the present invention.

Accordingly, the present invention is directed to composition(s) for reducing pain (such as, but not limited to, body pain).

The present invention is directed to composition(s) for reducing symptom(s) associated with seizures, slight strokes, and related illnesses and conditions.

The present invention is directed to method(s) for reducing pain (such as, but not limited to, body pain).

The present invention is directed to method(s) for reducing symptom(s) associated with seizures, slight strokes, and related illnesses and conditions.

The terms “externally” and “external” mean outside of the body part or area of the subject or user and on the skin adjacent to or in the proximity of the body part or area.

### Exemplary Embodiment 1

Composition mixture of ingredients:

approximately 2.0 grams to approximately 2.2 grams (preferably approximately 2.2 grams) of marijuana (from the cannabis plant, such as, but not limited to, Cannabis sativa or Cannabis indica plant);

approximately 1.5 ounces of hand salve (such as, but not limited to, Burt's Bees Hand Salve—such as from a 3 ounce/oz. tin of Burt's Bees Hand Salve);

approximately 1.5 ounces of healing ointment (such as, but not limited to, Aquaphor® healing ointment—such as from a 7 ounce/oz. tube of Aquaphor® healing ointment “Advanced Therapy”);

approximately 1.5 pints of water; and

approximately 2.0 ounces of oil (such as, but not limited to, coconut oil, vegetable oil, palm oil, sunflower oil, corn oil, canola oil, and peanut oil; preferably coconut oil).

Preferred composition mixture of ingredients:

approximately 2.2 grams of marijuana (from the cannabis plant, such as, but not limited to, Cannabis sativa or Cannabis indica plant);

approximately 1.5 ounces of Burt's Bees Hand Salve (such as from a 3 ounce/oz. tin of Burt's Bees Hand Salve);

approximately 1.5 ounces of Aquaphor® healing ointment (such as from a 7 ounce/oz. tube of Aquaphor® healing ointment “Advanced Therapy”);

approximately 1.5 pints of water; and

approximately 2.0 ounces of coconut oil.

### Exemplary Embodiment 2

Subject or user #1: 53 year old female with slight stroke symptom(s) and a lot of pain (such as body pain).
Composition mixture: from Exemplary Embodiment 1.
Administration or application method: apply (such as, but not limited to, rub) an effective amount of the composition mixture externally onto the subject's affected body part or area (but sometimes, alternatively or also, the head, neck, shoulders, back, legs, and arms) for about 3 days to about 7 days, and for 1-2 times (preferably 2 times) per day.
Result(s): about 80% to about 90% of pain and symptom(s) reduced. No side effect and no after effect observed.

### Exemplary Embodiment 3

Subject or user #2: 54 year old female with slight stroke symptom(s) and a lot of pain (such as body pain).
Composition mixture: from Exemplary Embodiment 1.
Administration or application method: apply (such as, but not limited to, rub) an effective amount of the composition mixture externally onto the subject's affected body part or area (but sometimes, alternatively or also, the head, neck, shoulders, back, legs, and arms) for about 3 days to about 7 days, and for about 1-2 times (preferably 2 times) per day.
Result(s): about 80% to about 90% of pain and symptom(s) reduced. No side effect and no after effect observed.

### Exemplary Embodiment 4

Subject or user #3: 74 year old male with slight stroke symptoms and a lot of pain (such as body pain).
Composition mixture: from Exemplary Embodiment 1.
Administration or application method: apply (such as, but not limited to, rub) an effective amount of the composition mixture externally onto the subject's affected body part or area (but sometimes, alternatively or also, the head, neck, shoulders, back, legs, and arms) for about 3 days to about 7 days, and for about 1-2 times (preferably 2 times) per day.
Result(s): about 80% to about 90% of pain and symptom(s) reduced. No side effect and no after effect observed.

### Exemplary Embodiment 5

Subject or user #4: 47 year old female with fits or seizures and convulsions for 3 years and a lot of pain (such as body pain).
Composition mixture: from Exemplary Embodiment 1.
Administration or application method: apply (such as, but not limited to, rub) an effective amount of the composition mixture externally onto the subject's affected body part or area (but sometimes, alternatively or also, the head, neck, shoulders, back, legs, and arms) for about 3 days to about 7 days, and for about 1-2 times (preferably 2 times) per day.
Result(s): about 80% to about 90% of pain and symptom(s) reduced. No side effect and no after effect observed.

### Exemplary Embodiment 6

Subject or user #5: about 50 year old male cyclist with pain (such as body pain) from a fall.
Composition mixture: from Exemplary Embodiment 1.
Administration or application method: apply (such as, but not limited to, rub) an effective amount of the composition mixture externally onto the subject's body part(s) that was experiencing pain for about 3 days to about 7 days, and for about 1-2 times (preferably 2 times) per day.
Result(s): about 80% to about 90% of pain reduced. No side effect and no after effect observed.

### Exemplary Embodiment 7

Method(s) for reducing pain and symptom(s) associated with seizures, slight strokes, and related illnesses and conditions comprising the step of:
providing an effective amount of a composition mixture of ingredients for being applied externally onto affected body part(s) or area(s) (but sometimes, alternatively or also, the head, neck, shoulders, back, legs, and arms) of a subject or user (such as those described in Exemplary Embodiments 2, 3, 4, 5 and 6), for about 3 days to about 7 days and for about 1-2 times (preferably 2 times) per day,
wherein the composition mixture comprises:

approximately 2.0 grams to approximately 2.2 grams (preferably approximately 2.2 grams) of marijuana (from the cannabis plant, such as, but not limited to, Cannabis sativa or Cannabis indica plant),

approximately 1.5 ounces of hand salve (such as, but not limited to, Burt's Bees Hand Salve—such as from a 3 ounce/oz. tin of Burt's Bees Hand Salve),

approximately 1.5 ounces of healing ointment (such as, but not limited to, Aquaphor® healing ointment—such as from a 7 ounce/oz. tube of Aquaphor® healing ointment “Advanced Therapy”),

approximately 1.5 pints of water, and

approximately 2.0 ounces of oil (such as, but not limited to, coconut oil, vegetable oil, palm oil, sunflower oil, corn oil, canola oil, and peanut oil; preferably coconut oil).

Preferred composition mixture of ingredients in this embodiment:

approximately 2.2 grams of marijuana (from the cannabis plant, such as, but not limited to, Cannabis sativa or Cannabis indica plant);

approximately 1.5 ounces of Burt's Bees Hand Salve (such as from a 3 ounce/oz. tin of Burt's Bees Hand Salve);

approximately 1.5 ounces of Aquaphor® healing ointment (such as from a 7 ounce/oz. tube of Aquaphor® healing ointment “Advanced Therapy”);

approximately 1.5 pints of water; and

approximately 2.0 ounces of coconut oil.

Result(s): about 80% to about 90% of pain and symptom(s) reduced. No side effect and no after effect observed.

Alternative to applying an effective amount of the composition mixture externally onto affected body part(s) or area(s) (but sometimes, alternatively or also, the head, neck, shoulders, back, legs, and arms) of a subject or user (such as those described in Exemplary Embodiments 2, 3, 4, 5 and 6), for about 3 days to about 7 days and for about 1-2 times (preferably 2 times) per day, the inventor believes that applying an effective amount of the composition mixture externally, as needed, onto affected body part(s) or area(s) (but sometimes, alternatively or also, the head, neck, shoulders, back, legs, and arms) of a subject or user (such as those described in Exemplary Embodiments 2, 3, 4, 5 and 6) will be sufficient to reduce pain (such as, but not limited to, body pain) and symptom(s) associated with seizures, slight strokes, and related illnesses and conditions.

It is to be understood that the present invention is not limited to the embodiments and/or aspects described above, but encompasses any and all embodiments and/or aspects within the spirit of the invention.

1. A composition for reducing pain comprising:
approximately 2.0 grams to approximately 2.2 grams of marijuana;
approximately 1.5 ounces of hand salve;
approximately 1.5 ounces of healing ointment;
approximately 1.5 pints of water; and
approximately 2.0 ounces of oil.

2. The composition according to claim 1, wherein said marijuana is approximately 2.2 grams.

3. The composition according to claim 1, wherein said marijuana is from a cannabis plant.

4. The composition according to claim 1, wherein said hand salve is Burt's Bees Hand Salve.

5. The composition according to claim 1, wherein said healing ointment is Aquaphor® healing ointment.

6. The composition according to claim 1, wherein said oil is coconut oil.

7. The composition according to claim 1, wherein said hand salve is Burt's Bees Hand Salve, said healing ointment is Aquaphor® healing ointment, and said oil is coconut oil.

8. A composition for reducing symptom(s) associated with seizures, slight strokes, and related illnesses and conditions, said composition comprising:
approximately 2.0 grams to approximately 2.2 grams of marijuana;
approximately 1.5 ounces of hand salve;
approximately 1.5 ounces of healing ointment;
approximately 1.5 pints of water; and
approximately 2.0 ounces of oil.

9. The composition according to claim 8, wherein said marijuana is approximately 2.2 grams.

10. The composition according to claim 8, wherein said marijuana is from a cannabis plant.

11. The composition according to claim 8, wherein said hand salve is Burt's Bees Hand Salve.

12. The composition according to claim 8, wherein said healing ointment is Aquaphor® healing ointment.

13. The composition according to claim 8, wherein said oil is coconut oil.

14. The composition according to claim 8, wherein said hand salve is Burt's Bees Hand Salve, said healing ointment is Aquaphor® healing ointment, and said oil is coconut oil.

15. A method for reducing pain, the method comprising the step of:
providing an effective amount of a composition for being applied externally, as needed, onto affected body part(s) or area(s) of a subject or user,
wherein the composition comprises approximately 2.0 grams to approximately 2.2 grams of marijuana, approximately 1.5 ounces of hand salve, approximately 1.5 ounces of healing ointment, approximately 1.5 pints of water, and approximately 2.0 ounces of oil.

16. The method according to claim 15, wherein the marijuana is approximately 2.2 grams.

17. The method according to claim 15, wherein the marijuana is from a cannabis plant.

18. The method according to claim 15, wherein the hand salve is Burt's Bees Hand Salve.

19. The method according to claim 15, wherein the healing ointment is Aquaphor® healing ointment.

20. The method according to claim 15, wherein the oil is coconut oil.

21. The method according to claim 15, wherein the hand salve is Burt's Bees Hand Salve, the healing ointment is Aquaphor® healing ointment, and the oil is coconut oil.

22. The method according to claim 15, wherein the effective amount of the composition is applied externally onto affected body part(s) or area(s) of the subject or user for about 3 days to about 7 days.

23. The method according to claim 15, wherein the effective amount of the composition is applied externally onto affected body part(s) or area(s) of the subject or user for 1-2 times per day.

24. The method according to claim 15, wherein the effective amount of the composition is applied externally onto affected body part(s) or area(s) of the subject or user for about 3 days to about 7 days and for 1-2 times per day.

25. A method for reducing symptom(s) associated with seizures, slight strokes, and related illnesses and conditions, the method comprising the step of:
providing an effective amount of a composition for being applied externally, as needed, onto affected body part(s) or area(s) of a subject or user,
wherein the composition comprises approximately 2.0 grams to approximately 2.2 grams of marijuana, approximately 1.5 ounces of hand salve, approximately 1.5 ounces of healing ointment, approximately 1.5 pints of water, and approximately 2.0 ounces of oil.

26. The method according to claim 25, wherein the marijuana is approximately 2.2 grams.

27. The method according to claim 25, wherein the marijuana is from a cannabis plant.

28. The method according to claim 25, wherein the hand salve is Burt's Bees Hand Salve.

29. The method according to claim 25, wherein the healing ointment is Aquaphor® healing ointment.

30. The method according to claim 25, wherein the oil is coconut oil.

31. The method according to claim 25, wherein the hand salve is Burt's Bees Hand Salve, the healing ointment is Aquaphor® healing ointment, and the oil is coconut oil.

32. The method according to claim 25, wherein the effective amount of the composition is applied externally onto affected body part(s) or area(s) of the subject or user for about 3 days to about 7 days.

33. The method according to claim 25, wherein the effective amount of the composition is applied externally onto affected body part(s) or area(s) of the subject or user for 1-2 times per day.

34. The method according to claim 25, wherein the effective amount of the composition is applied externally onto affected body part(s) or area(s) of the subject or user for about 3 days to about 7 days and for 1-2 times per day.
